(19) United States
(12) Patent Application Publication
(10) Pub. No.: US 2010/0016790 A1
(43) Pub. Date: Jan. 21, 2010

(54) Treatment And Pre-Treatment Device, And Manufacturing Method Therefor, Involving Nitric Oxide

(76) Inventor: Tor Peters, Helsingborg (SE)

Correspondence Address: INSKEEP INTELLECTUAL PROPERTY GROUP, INC, 2281 W. 190TH STREET, SUITE 200, TORRANCE, CA, 90504, US

(21) Appl. No.: 11/912,662

(22) PCT Filed: Feb. 13, 2006
(86) PCT No.: PCT/EP06/50899

§ 371 Date: Jun. 13, 2008

Related U.S. Application Data
(60) Provisional application No. 60/688,072, filed on Jun. 2, 2005.
(60) Provisional application No. 60/711,006, filed on Aug. 24, 2005.

(30) Foreign Application Priority Data
Jun. 1, 2005 (EP) 05011786.0
Aug. 23, 2005 (EP) 05018269.0

(51) Int. Cl. A61M 5/19; A61K 9/70; A61K 47/34; A61K 33/00; A61K 47/38; A61K 47/32; A61P 9/00

(52) U.S. Cl. 604/82; 424/443; 514/772.7; 424/718; 514/781; 514/772.3; 514/772.4; 424/445; 424/400

(57) ABSTRACT

A device is provided that allows for treatment or pre-treatment of an area of a human or animal organ intended to be penetrated to connect the vascular system of said human or animal with a sampling, infusion, or withdrawal container. The device comprises nitric oxide (NO) for obtaining a vaso-dilating effect at said area during said treatment or pre-treatment.

### RELATED APPLICATIONS

This application claims priority to International Application No. PCT/EP2006/050899 filed Feb. 13, 2006 entitled Treatment And Pre-Treatment Device, And Manufacturing Method Therefor, Involving Nitric Oxide, which claims priority to European Patent Application No. 05018269.0 filed Aug. 23, 2005 entitled Device, System, And Method Comprising Microencapsulated Liquid For Release Of Nitric Oxide From A Polymer; U.S. Provisional Application No. 60/711,006 filed Aug. 24, 2005 entitled Device, System, And Method Comprising Microencapsulated Liquid For Release Of Nitric Oxide From A Polymer; European Patent Application No. 05011786.0 filed Jun. 1, 2005 entitled Pre-Treatment Device; and U.S. Provisional Application No. 60/688,072 filed Jun. 2, 2005 entitled Pre-Treatment Device, all of which applications are hereby incorporated by reference.

### FIELD OF THE INVENTION

This invention pertains in general to the field of a device configured for preparing a subcutaneous tissue to insertion of a catheter, Venflon®, needle and/or syringe. More particularly the invention relates to a device for preparing a subcutaneous tissue to insertion of a catheter, vascular access devices, needle, and/or syringe and a process for manufacturing of said device, involving the use of nitric oxide (NO).

### BACKGROUND OF THE INVENTION

Catheters, Venflons®, needles, and/or syringes are well known for being used to fluidly communicate with the vascular system of a patient in a minimally invasive procedure, whether to withdraw fluids from the patient or to infuse fluids into the patient.

Venflons® and catheters are generally short thin flexible tubes that are open at a distal end and secured within a hub at a proximal end. The hub serves as a quick disconnectable mechanical connector between the vascular access devices or catheter and a delivery tube extending, for example, from a liquid source or a liquid withdrawal source.

Needles and syringes are unflexible, preferably made of a metallic material, devices with a tubular part, which are used to assist in application of catheters and Venflons®, according to below, and direct sampling, infusion, and withdrawal of body fluids.

One typical catheter is an over-the needle style catheter that requires an insertion needle passing there through to penetrate the patient's skin and advance the catheter into the patient's vascular system. The needle comprises a bevelled distal end to facilitate piercing the patient's skin.

During the insertion of the devices according to above it is often very complicated for the nurse or physician to find a suitable vessel in connection to the vascular system of the patient. This complication is caused by low vaso-dilation in the subcutaneous tissue in the target area.

Also, in the field of this technology, nurses and physicians, normally disinfect an area that is to be penetrated by the vascular access devices, catheter, needle, or syringe. This is usually done by rubbing said area with a cotton pad supplied with some kind of alcohol.

It is known that nitric oxide (NO) provides an alternative to conventional therapies, such as antibiotics. Nitric oxide is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc., and general microbial invasion. This improvement of healing is partly caused by NO inhibiting the activation or aggregation of blood platelets, and also by NO causing a reduction of inflammatory processes at the site of an implant.

NO is also known to have an anti-pathogenic, especially an anti-viral, effect, and furthermore NO has an anti-cancerous effect, as it is cytotoxic and cytostatic in therapeutic concentrations, i.e. it has among other effects tumoricidal and bacteriocidal effects. NO has for instance cytotoxic effects on human haematological malignant cells from patients with leukaemia or lymphoma, whereby NO may be used as a chemotherapeutic agent for treating such haematological disorders, even when the cells have become resistant to conventional anti-cancer drugs. This anti-pathogenic and anti-tumour effect of NO is taken advantage of by the present invention, without having adverse effects as for instance many anti-cancer drugs.

However, due to the short half-life of NO, it has hitherto been very hard to treat viral, bacteria, virus, fungi or yeast infections with NO. This is because NO is actually toxic in high concentrations and has negative effects when applied in too large amounts to the body. NO is actually also a vasodilator, and too large amounts of NO introduced into the body will cause a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

In recent years research has been directed to polymers with the capability of releasing nitrogen oxide when getting in contact with water. Such polymers are for example polyalkyleneimines, such as L-PEI (Linear PolyEthylenelmine) and B-PEI (Branched PolyEthylenelmine), which polymers have the advantage of being biocompatibleoxide.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner to tissues and organs to aid the healing process and to prevent injury to tissues at risk of injury. Electrospun nano-fibers of linear poly(ethylenimine) diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device.

However, the disclosure is both silent concerning an improvement of present technology in respect of a device for pretreatment of a subcutaneous area, that is to be penetrated by a vascular access devices, catheter, needle, or syringe, to increase vaso-dilation, and decrease contraction and spasm, and simultaneously provide an anti-bacterial and ant-viral effect, by elution of nitric oxide NO).

Hence, an improved device, or more advantageous, for pretreatment of a subcutaneous area, that is to be penetrated by a vascular access devices, catheter, needle, or syringe, is needed in the art. I is desired that said device does increase circulation in said area, has a vaso-dilating effect, is easy to use, does not develop resistance against the active pharmaceutical substance, and provides anti-microbial and anti-viral effect, etc, would be advantageous, and in particular a device allowing for facilitating insertion of Venflons®, catheters, needles, and syringes, etc., would be advantageous.

### SUMMARY OF THE INVENTION

Accordingly, the present invention preferably seeks to mitigate, alleviate or eliminate one or more of the above-identified deficiencies in the art and disadvantages singly or in any combination and solves among others at least the problems mentioned above, at least partly by providing a device according to the appended patent claims.

According to one aspect of the invention, a device is provided that allows for treatment and/or pre-treatment of an area of a human or animal organ, before, during, and/or after penetration of said area to connect the vascular system of said human or animal with a sampling, infusion, or withdrawal container. Said device comprises a nitric oxide (NO) eluting polymer arranged to contact said tissue, such that a therapeutic dose of nitric oxide is eluted from said nitric oxide eluting polymer to said tissue, allowing for increased vaso-dilation, decreased contraction and spasm, and anti-microbial and anti-viral effect.

The organ according to the present invention may for example be the skin on the head, face, neck, shoulder, back, arm, hand, stomach, genital, thigh, leg, or foot, of a body of said human or animal.

According to another aspect of the invention, a manufacturing process for such a device is provided, wherein the process is a process for forming a device that allows for pre-treatment of an area of a human or animal organ, which organ is to be penetrated to connect the vascular system of said human or animal with a sampling, infusion, or withdrawal container. The process comprises selecting a plurality of nitric oxide eluting polymeric fibers, and deploying said nitric oxide eluting fibers in a patch/pad, dressing, tape/coating, plaster/sheath, gel, hydrogel, foam, cream, etc., to be comprised in said device.

The present invention has at least the advantage over the prior art that it provides target exposure of an organ area to NO, whereby an increased circulation in the organ area, a vaso-dilating effect, a decreased contraction and spasm, anti microbial and anti-viral effect, while not developing resistance against the active pharmaceutical substance, local skin irritation, pain etc, are simultaneously obtained.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a patch/pad 10 according to an embodiment of the invention,

FIG. 2 is a schematic illustration of a tape or coating 20 according to an embodiment of the invention,

FIG. 3 is a schematic illustration of a sheath or plaster 30 according to an embodiment of the invention, and

FIG. 4 is an illustration of two elution profiles of two mixtures of nitric oxide eluting polymer and carrier material.

### DESCRIPTION OF EMBODIMENTS

The following description focuses on embodiments of the present invention applicable to a device, in form of a patch/pad, dressing, gel, hydrogel, foam, cream, tape/coating, etc., which allows for treatment and/or pretreatment of an area of a human or animal organ, before, during, and/or after penetration of said area, to connect the vascular system of said human or mammal with a sampling, infusion, or withdrawal container, as well as a manufacturing method for the latter and a use of nitric oxide. This sampling, infusion, or withdrawal container may for example be, or be in communication with, or connected to, a catheter, vascular access devices, syringe, or needle, but the sampling, infusion, or withdrawal container according to the present invention is not intended to be limited to these examples. These examples are only intended to be illustrative in respect of the present invention. The registered trademark vascular access devices is used in the present invention not to limit the scope of the present invention but merely to give an example of what devices are included, and all devices functioning in the same way as vascular access devices are also within the scope of the present invention.

The animal according to the present invention may for example be selected from any mammal, such as cat, dog, horse, cattle, bird, pig, etc., or any other possible animal with a vascular system.

With regard to nitric oxide (nitrogen monoxide, NO), its physiological and pharmacological roles have attracted much attention and thus have been studied. NO is synthesized from arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is 2 to 3 orders higher, i.e. 100 to 1000 folded higher, and that iNOS produces an extremely large amount of NO.

In the case of the generation of a large amount of NO as in the case of the production by iNOS, it is known that NO reacts with active oxygen to attack exogenous microorganisms and cancer cells, but also to cause inflammation and tissue injury. On the other hand, in the case of the generation of a small amount of NO as in the case of the production by cNOS, it is considered that NO takes charge of various protective actions for a living body through cyclic GMP (cGMP), such as vasodilator action, improvement of the blood circulation, antiplatelet-aggregating action, antibacterial action, anticancer action, acceleration of the absorption at the digestive tract, renal function regulation, neurotransmitting action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

In recent years research has been directed to polymers with the capability of releasing nitrogen oxide when getting in contact with water. Such polymers are for example polyalkyleneimines, such as L-PEI (Linear PolyEthylenelmine) and B-PEI (Branched PolyEthylenelmine), which polymers have the advantage of being biocompatible.

The polymers employed in embodiments of the present invention may be manufactured by electro spinning, air spinning, gas spinning, wet spinning, dry spinning, melt spinning, and gel spinning. Electro spinning is a process by which a suspended polymer is charged. At a characteristic voltage a fine jet of polymer releases from the surface in response to the tensile forces generated by interaction by an applied electric field with the electrical charge carried by the jet. This process produces a bundle of polymer fibres, such as nano-fibres. This jet of polymer fibres may be directed to a surface to be treated.

Furthermore, U.S. Pat. No. 6,382,526, U.S. Pat. No. 6,520,425, and U.S. Pat. No. 6,695,992 disclose processes and apparatuses for the production of such polymeric fibres. These techniques are generally based on gas stream spinning, also known within the fiber forming industry as air spinning, of liquids and/or solutions capable of forming fibers. Gas stream spinning is suited for producing devices according to certain embodiments of the invention.

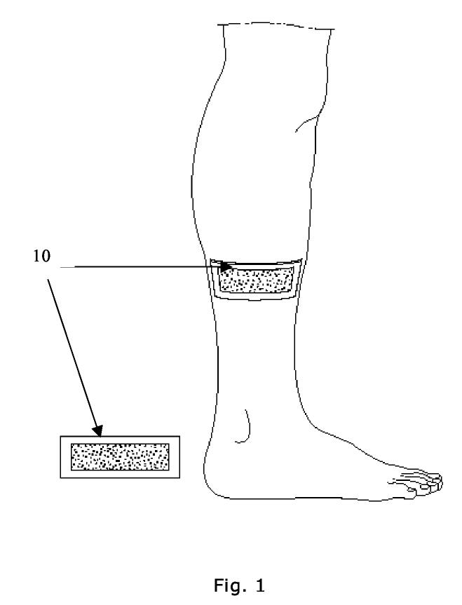

In an embodiment of the invention, according to FIG. 1, the device according to the present invention is in patch/pad, manufactured of a combination of L-PEI or other NO-eluting polymer, such as amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone, and other suitable carrier materials, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these, as base material, where NO is allowed to be eluted, said patch/pad being covered on the inside with nano-filament of NO-eluting L-PEI. The base material of the patch/pad according to the present invention may also be cotton, polyacrylate or any other fabric used in the clothing industry, in which cases the base material is loaded with the NO-eluting polymer according to the invention. This embodiment provides an easy to use patch/pad, which is applied on the area to be penetrated by a catheter, vascular access devices, syringe, or needle.

Three important factors in controlling and regulating the elution of nitric oxide from a nitric oxide eluting polymer are how quickly a proton donor comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

In one embodiment of the present invention a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of an hydrophilic polymer is polyethylene oxide, and one example of an hydrophobic polymer is polystyrene. These carrier polymers may be mixed with the nitric oxide eluting polymer and then electrospun to suitable fibers. The skilled person in the art knows which other polymers may be used for similar purposes. FIG. 4 illustrates two elution profiles (NO concentration vs. time) for two different polymer mixtures; a nitric oxide eluting polymer mixed with a hydrophilic carrier polymer in an acidic environment (A), and a nitric oxide eluting polymer mixed with a hydrophobic carrier polymer in a neutral environment (B).

In one embodiment of the present invention the device is configured to treat and/or pre-treat an area of a human or animal organ before, during, and/or after penetration of said area, to connect the vascular system of said human or animal with a sampling, infusion, or withdrawal container. Said device is configured to elute nitric oxide (NO) to obtain a vaso-dilating, anti-contraction and anti-spasm effect at said area. Means, such as encapsulated water, for initiating elution of nitric oxide may be integrated in said device.

When the patch/pad according to an embodiment of the present invention gets in contact with the skin, and thereby gets in contact with the moisture in form of secreted sweat, the NO-eluting patch/pad starts to release NO to said area.

The thus eluted NO has a vasodilating effect and anti-contraction and anti-spasm effect on the area, which effect results in that the blood vessels in said area will expand and the risk of spasm of the blood vessel is decreased. Spasm of the blood vessel is a common phenomena during penetration, which phenomena makes penetration difficult. Furthermore, expanded blood vessels are easier to locate, which make it easier to the nurse or physician to choose which blood vessel to insert the catheter, vascular access devices, syringe, or needle, in. It is also much easier to the nurse or physician to penetrate the blood vessel with said catheter, vascular access devices, syringe, or needle, when the blood vessel is expanded.

NO has not only a vasodilating effect but also provides an anti-microbial and anti-viral effect. Thus, there is no need to disinfect the area, intended to be subjected to insertion of a catheter, vascular access devices, syringe, or needle, with for example alcohol, which is common practice in the caretaking of today.

In another embodiment of the present invention the patch/pad is covered on the inside with nano-filament of any other suitable polymer, according to above. Such polymers are for example other polyalkyleneimines, such as B-PEI (Branched PolyEthylenelmine) or PEI-cellulose, which polymers have the advantage of being biocompatible.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner.

However, the meaning of “controlled” in the context of U.S. Pat. No. 6,737,447 is only directed to the fact that nitric oxide is eluted from the coating during a period of time, i.e that the nitric oxide not is eluted all in once. Therefore, the interpretation of “controlled” in respect of U.S. Pat. No. 6,737,447 is different from the meaning of “regulating” in the present invention. “Regulate or control”, according to the present invention is intended to be interpreted as the possibility to vary the elution of nitric oxide to thereby achieve different elution profiles.

A polymer comprising an O-nitrosylated group is also a possible nitric oxide eluting polymer. Thus, in one embodiment of the present invention, the nitric oxide eluting polymer comprises diazeniumdiolate groups, S-nitrosylated and O-nitrosylated groups, or any combinations thereof.

In still another embodiment of the present invention said nitric oxide eluting polymer is a poly(alkyleneimine)diazeniumdiolate, such as L-PEI-NO (linear poly(ethyleneimine)diazeniumdiolate), where said nitric oxide eluting polymer is loaded with nitric oxide through the diazeniumdiolate groups and arranged to release nitric oxide at a treatment site.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

In still another embodiment of the present invention the nitric oxide eluting polymer may be a O-derivatized NONOate. This kind of polymer often needs an enzymatic reaction to release nitric oxide.

Other ways of describing polymers, which may be suitable as nitric oxide eluting polymer, is polymers comprising secondary amine groups (═N—H), such as L-PEI, or have a secondary amine (═N—H) as a pendant, such as aminocellulose.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbour atom, such as a carbon atom, to the nitrogen atom, with higher electronegativity than nitrogen (N), it is very difficult to load the polymer with nitric oxide. On the other hand, if there is a electropositive ligand close to the secondary amine, such as on a neighbour atom, such as a carbon atom, to the nitrogen atom, the electronegativity of the amine will increase and thereby increase the possibility to load the nitric oxide elution polymer with nitric oxide.

In an embodiment of the present invention the nitric oxide polymer may be stabilized with a salt. Since the nitric oxide eluting group, such as a diazeniumdiolate group, usually is negative, a positive counter ion, such as a cation, may be used to stabilize the nitric oxide eluting group. This cation may for example be selected from the group comprising any cation from group 1 or group 2 in the periodic table, such as Na⁺, K⁺, Li⁺, Be²⁺, Ca²⁺, Mg²⁺, Ba²⁺, and/or Sr²⁺. Different salts of the same nitric oxide eluting polymer have different properties. In this way a suitable salt (or cation) may be selected for different purposes. Examples of cationic stabilized polymers are L-PEI-NO—Na, i.e. L-PEI diazeniumdiolate stabilized with sodium, and L-PEI-NO—Ca, i.e. L-PEI diazeniumdiolate stabilized with calcium.

Another embodiment of the present invention comprises mixing the nitric oxide eluting polymer, or a mixture of the nitric oxide eluting polymer and a carrier material, with an absorbent agent. This embodiment provides the advantage of an accelerated elution of nitric oxide since the polymer, or polymer mixture, via the absorbent agent, may take up the activating fluid, such as water or body fluid, much faster. In one example 80% (w/w) absorbent agent is mixed with the nitric oxide eluting polymer, or mixture of nitric oxide eluting polymer and carrier material, and in another embodiment 10 to 50% (w/w) absorbent agent is mixed with the nitric oxide eluting polymer, or mixture of nitric oxide eluting polymer and carrier material.

Since the elution of nitric oxide is activated by a proton donor, such as water, it may be an advantage to keep the nitric oxide eluting polymer, or mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. Therefore, in still another embodiment of the present invention, the elution of nitric oxide may be regulated by adding an absorbent agent. The absorbent agent absorbs the proton donor, such as water, and keeps the proton donor in close contact with the nitric oxide eluting polymer during prolonged periods of time. Said absorbent agent may be selected from the group comprising polyacrylates, polyethylene oxide, carboxymethylcellulose, and microcrystalline cellulose, cotton, and starch. This absorbent agent may also be used as a filling agent. In this case said filling agent may give the nitric oxide eluting polymer, or mixture of said nitric oxide eluting polymer and a carrier material, a desired texture.

Thus, it is most preferable that the nano-spun fibres in the NO-eluting patch/pad according to the present embodiment of the present invention comprise PEI. Also nano-filaments to be woven into the patch/pad are suitably produced from PEI and loaded with NO for release thereof at use.

In another embodiment of the present invention the patch/pad according to the present invention is covered on the inside with NO-eluting nano-particles, or micro-spheres. These nano-particles, or micro-spheres, may be formed from the NO-eluting polymers according to the present invention, encapsulated in any suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. When the nano-particles, or micro-spheres, according to this embodiment, gets in contact with the secreted moisture, in form of sweat, on the inside of the patch/pad, they start to elute NO on the area to be pre-treated.

In yet another embodiment of the present invention the patch/pad contains a small water bag or sealed water sponge. This water bag or sealed water sponge is used to activate the elution of NO from the NO-eluting polymer, nano-particles, and/or micro-spheres. Persons that not easily sweat may be helped by the use of this embodiment. Alternatively, the patch/pad may be soaked with water after, or just before, it is applied on said area. This bag or sponge may also contain other proton donors, which proton donors are listed below.

In another embodiment of the present invention a nitric oxide eluting polymer is provided, and/or combined, with microencapsulated proton donor.

This may for example be done by first manufacture micro capsules, containing a proton donor, such as water or water containing liquid, in a state of the art manner. These micro capsules are then applied on the NO eluting polymer. The application of the micro capsules on the NO eluting polymer may for example be done by gluing, such as pattern gluing, or instead spinning the NO eluting polymer onto said micro capsules. In this way a device or a system, comprising NO eluting polymer and micro encapsulated proton donor is manufactured. When the device or system is applied on the target area the device or system is compressed or squeezed. Said compression or squeezing results in breakage of the micro capsules. The NO eluting polymer is thus exposed to proton donor, and the elution of NO from the NO eluting polymer is initiated on the target area. In other embodiments of the present invention the proton donor inside the micro capsules is released by heating or shearing the micro capsules until the micro capsules are ruptured.

In still another embodiment the micro capsules are formed into a film, tape, or sheath. Thereafter, a film, tape, or sheath of an NO eluting polymer is glued onto the film, tape, or sheath of micro capsules. Preferably the film, tape, or sheath of the NO eluting polymer is glued onto the film, tape, or sheath of the micro capsules in patterned way. The obtained pattern includes spaces where there is no glue, in which spaces the proton donor will be transported to the NO eluting polymer once the micro capsules are broken from compression or squeezing. When the proton donor gets in contact with the NO eluting polymer the elution of NO starts. Thus, the combination of film, tape, or sheath of micro capsules and NO eluting polymer may be applied on a target area. Thereafter the combination is compressed or squeezed, which results in that the target area is exposed to NO.

I yet another embodiment the NO eluting polymer is spun directly onto the film, tape, or sheath of micro capsules, containing proton donor. The combination of film, tape, or sheath of micro capsules and spun NO eluting polymer may be applied on a target area. Thereafter the combination is compressed or squeezed, which results in that the target area is exposed to NO.

In still another embodiment of the present invention the device or system is provided with an activation indicator. This activation indicator indicates when the micro capsules are satisfyingly broken, hence when the NO eluting polymer is subjected to enough proton donor to elute an efficient amount of NO. This activation indicator may for example be obtained by colouring the proton donor that is trapped inside the micro capsules. When the micro capsules are broken the coloured proton donor escapes the microcapsules and the colour gets visualised while efficiently wetting the NO eluting polymer. Another way of obtaining an activation indicator is to choose to manufacture the micro capsules in a material, or choose a wall thickness of said micro particles, that creates a sound when the micro capsules break. It is also possible to admix a scent in the proton donor, contained in the micro capsules. This results in that the user of the device or system may smell the scent when the proton donor escapes from the micro capsules after breakage thereof.

In another embodiment a substance that changes color when it comes in contact with water can be incorporated in the device. Thus when the water capsules or water bag breaks the material changes color, thereby indicating that the material is activated.

In another embodiment of the present invention the device or system only allows NO-elution in one direction. In this kind of embodiment one side of the device has low permeability, or substantially no permeability, to nitric oxide. This may also be accomplished by applying a material on one side of the device according to the invention that is not permeable to NO. Such materials may be chosen from the group comprising common plastics, such as fluoropolymers, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, and latex, or any combinations of these. This embodiment is also easy to manufacture as the NO eluting polymer, e.g. L-PEI (or nitric oxide eluting polymer and carrier material, which will be explained in more detail below) may be electro or gas-jet spun onto the surface of the device according to the invention of e.g. the mentioned plastics, latex, or cotton.

In still another embodiment the device is provided with one membrane, which is permeable to nitric oxide, on a first side of the device, and another membrane, which has low permeability or substantially no permeability to nitric oxide, on a second side of said device. This embodiment provides the possibility to direct the elution to said first side of the device, while the elution of nitric oxide is substantially prevented from said second side. Thereby, a greater amount of nitric oxide will reach the intended area to be treated.

The activation of the nitric oxide eluting polymer may be accomplished by contacting said polymer with a suitable proton donor. In one embodiment the proton donor may be selected from the group comprising water, body fluids (blood, lymph, bile, etc.), alcohols (methanol, ethanol, propanols, buthanols, pentanols, hexanols, phenols, naphtols, polyols, etc.), aqueous acidic buffers (phosphates, succinates, carbonates, acetates, formats, propionates, butyrates, fatty acids, amino acids, etc.), or any combinations of these.

By adding a surfactant in the proton donor one can facilitate the wettening of the device. The surfactant lowers the surface tension and the activating fluid is easily transported throughout the device.

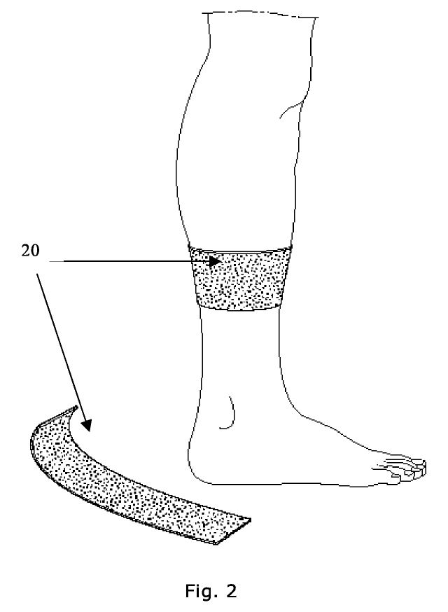

In still another embodiment of the device according to the present invention, it may be manufactured in the form of a polyurethane, or polyethylene, tape or coating, according to FIG. 2. This polyurethane tape or coating may easily be applied on the area intended to be subjected to insertion of a catheter, vascular access devices, syringe, or needle. At least the side facing the body part may be covered with NO-eluting nano-particles, micro-spheres, or nano-filament of NO-eluting L-PEI. When these particles or filaments get in contact with the moisture, in form of sweat, or proton donor, such as water, applied in any other way, such as spraying or bathing, on the inside of the tape or coating, the elution of NO starts.

This embodiment makes it possible to obtain a device that may be applied on locations that are difficult to get at with a patch/pad, such as in between the toes or fingers, the groin, the armpit etc.

In other embodiments of the invention, the tape/coating may be manufactured by any other suitable material, such as rubbers and plastics, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these, which material then is covered by an NO eluting polymer according to the present invention.

In another embodiment the nano-particles, or micro-spheres according to above, may be integrated in a soluble film that disintegrates on the inside of the patch/pad or tape/coating according to the present invention, in order to elute NO at the area of interest when the soluble film gets in contact with the moisture, in form of sweat or from the water bag or sealed water sponge, on the area to be treated.

When placed on an area to be pre-treated the device according to the present invention provides NO-elution, which results in vasodilating effect. This vasodilating effect expands the blood vessels, which expansion facilitate insertion of a catheter, vascular access devices, syringe, or needle in said blood vessel.

In another embodiment of the present invention the device only allows NO-elution in one direction. In this kind of embodiment one side of the patch/pad or tape/coating is non-permeable to NO. This may be accomplished by applying a material on one side of the patch/pad or tape/coating that is not permeable to NO. Such materials may be chosen from the group comprising common plastics, such as polyethylene, polyurethane, polyesters, polyamides, polyethers, polycarbonates, polyacrylonitrile, polystyrene, polypropylene, poly(acrylic acid) polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

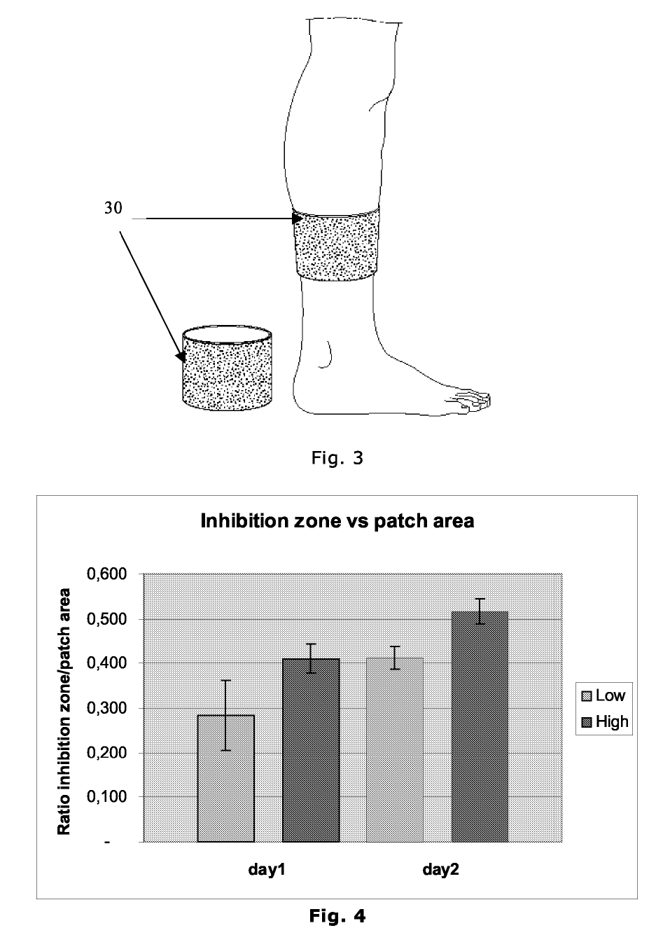

In another embodiment of the present invention, the device is in form of polyurethane or polyethylene sheaths or plasters, pads or dressings according to FIG. 3, coated with the NO-eluting polymer according to the present invention. The plaster or sheath may be applied on the area intended for insertion of a catheter, vascular access devices, syringe, or needle.

In other embodiments of the present invention the devices are covered with a powder, manufactured from nano-fibres of NO-eluting polymer, such as L-PEI, B-PEI, and/or PEI-cellulose. In this embodiments the devices according to the present invention are covered with said powder in the same way as the devices according to above were covered with nano-particles and/or micro-spheres.

In still another embodiment of the present invention the patch/pad, tape/coating, sheath/plaster, or dressing, according to above, is packaged in an air and/or light tight protective packaging. When one side of the protective packaging is removed a side covered with the NO eluting polymer according to the embodiments of the present invention is applied on the area to be pre-treated, on which area the device starts to elute NO.

In still another embodiment of the present invention the device is packaged in a protective packaging comprising a water bag, or other suitable water reservoir. Just before application of the device on the area to be pre-treated the water bag, or other suitable water reservoir, is broken. Thereafter the wetted device according to the present invention is applied on the area to be pre-treated, after which the device starts to elute NO.

In another embodiment of the device according to the present invention the fibres, nano-particles, micro-spheres, and/or powder may be integrated in a gel, that may either be in a smearing or compressed structure. The elution of NO may then be initiated by applying a water soaked patch on said gel. The fibres, nano-particles, or micro-spheres may also be integrated in a hydrogel, which is mixed directly before use. These embodiments have the advantage of being able to penetrate pockets and corners in the skin for closer elution of NO on the area to be pretreated. Since the nitric oxide eluting polymer is activated by proton donors the nitric oxide eluting polymer has to be separate from the proton donor until one wants to initiate the elution of nitric oxide, i.e. use the device. One way to accomplish this is to have a syringe with two separate containers. In one container you have a proton donor-based gel and in the other a non proton donor-based gel, comprising the nitric oxide eluting polymer. Upon using the device the two gels are squeezed from the syringe and mixed together, the proton donor in the first gel comes in contact with the nitric oxide eluting polymer in the second gel and the elution of nitric oxide starts.

In still another embodiment the nitric oxide eluting polymer, such as powder, nano-particles or micro-spheres, can be incorporated in foam. The foam may have an open cell structure, which facilitates the transport of the proton donor to the nitric oxide eluting polymer. The foam can be of any suitable polymer such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these, or latex.

In still another embodiment the device of the present invention is in form of a cream, or spray. The cream or spray may comprise the NO eluting polymer in a non aqueous solvent, such as an oil based solvent. First, the cream or spray is applied on the area to be pre-treated, then water, or another proton donor is applied to initiate the elution of NO. It is also possible to have a cream or spray comprising NO eluting polymer in a coating material according to above, which coating material breaks upon pressure, which breakage initiate elution of NO.

All embodiments of the present invention may be provided with an adhering material, such as a glue, etc., for facilitating the application of the devices on the area intended to be penetrated by the catheter, vascular access devices, syringe, needle, etc.

The device elutes nitric oxide (NO) from said eluting polymer in a therapeutic dose, such as between 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 1 to 1000 ppm.

In the embodiments of the present invention it may be suitable to control or regulate the time span of NO release from the device according to the invention. This may be accomplished by integrating other polymers or materials in said device. These polymers or materials may be chosen from any suitable material or polymer, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

The NO-eluting polymers in the devices according to the present invention may be combined with silver, such as hydroactivated silver. The integration of silver in the devices according to the present invention gives the anti-microbial and anti-viral effect an extra boost. Preferably the silver is releasable from the devices in the form of silver ions. The integration of silver in the device may present several advantages. One example of such an advantage is that the silver may keep the device in itself free from bacteria or viruses, while the nitric oxide eluting polymer elutes the therapeutic dosage of nitric oxide to the target site.

In yet another embodiment of the present invention the NO-eluting device is acting as a booster for drug eluting patches, e.g. pharmaceuticals, vitamins, nicotin, nitroglycerin, Non-Steroidal Anti-Inflammatory Drugs (NSAID), such as diclofenac, ibuprofen, aspirin, naproxen, COX-2 inhibitors, choline magnesium trisalicylate, diflunisal, salsalate, fenoprofen, flurbiprofen, ketoprofen, oxaprozin, indomethacin, sulindac, tolmetin, meloxicam, piroxicam, meclofenamate, mefenamic acid, nabumetone, etodalac, ketorolac, celecoxib, valdecoxib, and rofecoxib; steroids, such as cortisone, prednisone, methylprednisolone, prednisolone, vitamin D, estrogen, cholestrol, beclomethasone, flunisolide, fluticasone, triamcinolone, desonide, clobetasol, alclometasole, desoximetasone, betamethasone, halcinonide and dexamethasone; pain reliefs, such as motrin, feldene, naprosyn, lidocaine, and prilocalne; and other substances, such as indinavirsulfate, finasteride, aprepitant, montelukast sodium, alendronate sodium, rofecoxib, rizatriptan benzoate, simvastatin, finasteride, ezetimibe, caspofungin acetate, ertapenem sodium, dorzolamide hydrochloride, timolol maleate, losartan potassium, and hydrochlorotiazide; etc. This embodiment presents a device with the advantage of combining two treatments, of significant value, in one treatment.

The device according to the present invention may be manufactured by, for example electro spinning, gas spinning, air spinning, wet spinning, dry spinning, melt spinning, or gel spinning, of for example L-PEI. L-PEI is then, when manufactured by electro spinning, charged at a characteristic voltage, and a fine jet of L-PEI releases as a bundle of L-PEI polymer fibres. This jet of polymer fibres may be directed to a surface to be treated. The surface to be treated may for example be any suitable material in respect of a device according to the present invention. The electro spun fibres of L-PEI then attach on said material and form a coating/layer of L-PEI on the device according to the invention.

The basic material of the device according to the present invention may be polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. The NO-eluting polymer may be integrated in, spun together with, or spun on top of, any of these materials in all of the embodiments of the present invention.

It is of course possible to electro spin the other NO-eluting polymers, according to above, on the device according to the invention while still being inside the scope of the present invention.

In one embodiment the NO-eluting polymers employed in the devices according to the present invention are electro spun in such way that pure NO-eluting polymer fibres may be obtained.

Gas stream spinning, air spinning, wet spinning, dry spinning, melt spinning, and gel spinning, of said NO-eluting polymers onto the device according to the present invention is also within the scope of the present invention.

The manufacturing process according to the present invention presents the advantages of large contact surface of the NO-eluting polymer fibres or micro particles with the area to be pretreated, effective use of NO-eluting polymer, and a cost effective way of producing the device according to the present invention.

The invention may be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A device configured to expose a penetratable cutaneous area of a human or animal to nitric oxide (NO) before and/or during penetration of said penetratable cutaneous area in order to connect a vascular system of said human or animal with a sampling, infusion, or withdrawal container, wherein
said device is configured to elute said nitric oxide (NO) from said device substantially towards said penetratable cutaneous area for said exposure, in such a manner that said directed elution of said nitric oxide (NO) in use obtains a vasodilating, anti-contraction and anti-spasm effect at said penetratable cutaneous area.

2. The device according to claim 1, wherein said device facilitates insertion and/or penetration of a catheter, vascular access devices, syringe, or needle in a blood vessel of said vascular system at said penetratable cutaneous area by said vasodilating, anti-contraction and anti-spasm effect.

3. The device according to claim 2, wherein said device is devised to facilitate said insertion of said catheter, vascular access devices, syringe, or needle in said blood vessel by said vasodilating effect.

4. The device according to claim 1, wherein said device is devised to provide said vasodilating, anti-contraction and anti-spasm effect during said penetration.

5. The device according to claim 1, comprising a first membrane, which is permeable to nitric oxide, on a first side of the device, said first side in use is oriented towards said penetratable cutaneous area, and a second membrane, which has low permeability or substantially no permeability to nitric oxide, on a second side of said device, which in use is oriented away from said penetratable cutaneous area, such that said substantial direction of nitric oxide (NO) from said device in use thereof is provided as the elution of nitric oxide from said device in use is substantially prevented from said second side.

6. The device according to claim 1, wherein said device comprises a nitric oxide eluting polymer configured to elute a non-toxic dosage of nitrogen oxide (NO) when used for said exposure.

7. The device according to claim 6, wherein said nitric oxide (NO) eluting polymer comprises diazeniumdiolate groups, S-nitrosylated groups, and O-nitrosylated groups, or any combination of these.

8. The device according to claim 6, wherein said nitric oxide (NO) eluting polymer is L-PEI (linear polyethyleneimine), loaded with nitric oxide (NO) through said diazeniumdiolate groups, S-nitrosylated groups, or O-nitrosylated groups, or any combination of these, arranged for release of the nitric oxide (NO) at said penetratable cutaneous area.

9. Device according to claim 6, wherein said nitric oxide eluting polymer is selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

10. The device according to claim 1, wherein said device has a form selected from the group comprising of a patch/pad, a tape/coating, a dressing and a sheath/plaster.

11. The device according to claim 10, wherein said patch/pad, tape/coating, dressing, or sheath/plaster is manufactured from polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these, and said patch/pad, tape/coating, dressing, or sheath/plaster includes a nitric oxide (NO) eluting polymer configured for in use eluting said nitric oxide (NO) to said penetratable cutaneous area.

12. The device according to claim 1, wherein said device comprises means for initiating elution of said nitric oxide.

13. The device according to claim 12, wherein said means for initiating elution of nitric oxide is a proton donor bag, sealed proton donor sponge, or a microencapsulated proton donor.

14. The device according to claim 13, wherein said proton donor is selected from the group comprising water, blood, lymph, bile, methanol, ethanol, propanols, buthanols, pentanols, hexanols, phenols, naphtols, polyols, phosphates, succinates, carbonates, acetates, formats, propionates, butyrates, fatty acids, and amino acids, or any combinations of these.

15. The device according to claim 13, wherein said proton donor bag, sealed proton donor sponge, microencapsulated proton donor is included in a protective packaging of said device.

16. The device according to claim 1, wherein said device is packaged in a protective packaging prior to use.

17. The device according to claim 1, wherein said device is partly disintegrable when subjected to moisture or water.

18. The device according to claim 1, wherein said polymer comprises silver, configured for exposure of said area.

19. The device according to claim 1, wherein said polymer is in form of nano-particles or micro-spheres.

20. The device according to claim 19, wherein said nano-particles, or micro-spheres, are encapsulated in suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

21. The device according to claims 19, wherein said nano-particles, or micro-spheres, are integrated in a gel, hydrogel, foam, spray, or cream.

22. Device according to claim 1, wherein said device comprises a carrier material adapted to regulate or control said elution of said NO, selected from the group comprising polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these.

23. Device according to claim 6, wherein said NO-eluting polymer is applied on, or integrated with, a material selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these.

24. Device according to claim 6, wherein said nitric oxide eluting polymer comprises a secondary amine in the backbone or a secondary amine as a pendant.

25. Device according to claim 24, wherein a positive ligand is located on the neighbour carbon atom to the secondary amine.

26. Device according to claim 1, comprising an absorbent agent.

27. Device according to claim 26, wherein said absorbent agent is selected from the group comprising polyacrylate, polyethylene oxide, Carboxy Methyl Cellulose (CMC), microcrystalline cellulose, cotton, or starch, or any combinations thereof.

28. Device according to claim 6, comprising a cation, said cation stabilizing the nitric oxide eluting polymer.

29. Device according to claim 28, wherein said cation is selected from the group comprising Na⁺, K⁺, Li⁺, Be²⁺, Ca²⁺, Mg²⁺, Ba²⁺, and/or Sr²⁺, or any combinations thereof.

30. Device according to claim 22, wherein said carrier material is a hydrogel.

31. Device according to claim 6, wherein the nitric oxide eluting polymer is activatable by proton donors, wherein a the nitric oxide eluting polymer is, prior to use, stored separate from the proton donor until initiation of elution of nitric oxide therefrom.

32. Device according to claim 31, wherein the device is a syringe having two separate containers, wherein a first container contains a proton donor-based NO release activation agent, such as a gel, and a second container contains a non proton donor-based gel, comprising the nitric oxide eluting polymer, wherein the syringe is configured to provide admixing upon administration to said area.

33. A manufacturing process for a device according to claim 1, configured to expose a penetratable cutaneous area of a human or animal to nitric oxide (NO) before and/or during penetration of said penetratable cutaneous area in order to connect a vascular system of said human or animal with a sampling, infusion, or withdrawal container, comprising:
selecting a nitric oxide (NO) eluting material, such as an NO eluting polymer, configured to elute nitric oxide (NO) for said exposure,
selecting a carrier material, which carrier material is configured to regulate and control the elution of said nitric oxide (NO),
incorporating said NO-eluting material with said carrier material into an nitric oxide (NO) eluting material, such that said carrier material, in use of said device, regulates and controls the elution of said nitric oxide (NO), and
deploying said nitric oxide eluting material into a suitable form, or as a coating onto a carrier, to form at least a part of said device, such that said device is configured to expose said penetratable cutaneous area to said nitric oxide when said NO-eluting polymer in use elutes nitric oxide (NO).

34. The manufacturing process according to claim 33,
wherein said deploying comprises electro spinning, air spinning, gas spinning, wet spinning, dry spinning, melt spinning, or gel spinning of NO-eluting polymer or NO eluting material.

35. The manufacturing process according to claim 33, wherein said nitric oxide (NO) eluting material is a nitric oxide (NO) eluting polymer and said selecting comprises selecting a plurality of nitric oxide (NO) eluting polymeric particles, preferably nano fibres, nano particles or micro spheres.

36. The manufacturing process according to claim 33, wherein said nitric oxide (NO) eluting material is a NO-eluting polymer and said incorporating with said carrier material comprises integrating said NO-eluting polymer in said carrier material, spinning said NO-eluting polymer together with said carrier material, or spinning said NO-eluting polymer on top of said carrier material, in order to predefine nitric oxide eluting characteristics of said device.

37. The manufacturing process according to claim 33, further comprising integrating silver in said device.

38. The manufacturing process according to claim 33, further comprising microencapsulating a proton donor in micro capsules, and
applying the micro capsules to said nitric oxide (NO) eluting material.

39. The manufacturing process according to claim 38, wherein said applying comprises pattern gluing, or spinning the NO eluting material onto said micro capsules.

40. The manufacturing process according to claim 38, comprising forming the micro capsules into a first film, tape, or sheath,
forming a second film, tape, or sheath of said NO eluting material, and
gluing the first film, tape, or sheath of micro capsules to said second film, tape, or sheath of said NO eluting material.

41. The manufacturing process according to claim 40, wherein said gluing comprises patterned gluing, such that a pattern is obtained including glue free spaces.

42. The manufacturing process according to claim 38, comprising forming the micro capsules into a first film, tape, or sheath, and directly spinning the NO eluting material onto the film, tape, or sheath of micro capsules, containing a proton donor.

43. The manufacturing process according to claim 38, comprising providing an activation indicator configured to indicate when the micro capsules are broken such that the NO eluting material is subjected to said proton donor to elute NO.

44. The manufacturing process according to claim 43, wherein said providing an activation indicator comprises providing a coloring agent inside the micro capsules.

45. The manufacturing process according to claim 43, wherein said providing an activation indicator comprises selecting a material for the micro capsules, or choosing a wall thickness of said micro capsules, that creates a sound when the micro capsules break.

46. The manufacturing process according to claim 43, wherein said providing an activation indicator comprises admixing a scent material into the micro capsules.

47. The manufacturing process according to claim 43, wherein said providing an activation indicator comprises providing a substance that changes color when it comes in contact with the proton donor.

48. Use of a nitric oxide (NO) eluting polymer for the manufacture of a device according to claim 1, configured to expose a penetratable cutaneous area of a human or animal to nitric oxide (NO) before and/or during penetration of said penetratable cutaneous area in order to connect a vascular system of said human or animal with a sampling, infusion, or withdrawal container, wherein
nitric oxide is loaded to said device, which device such is configured to elute nitric oxide (NO) from said eluting polymer in a non-toxic dose when used on said penetratable cutaneous area,
for obtaining a vasodilating, anti-contraction and anti-spasm effect at said penetratable cutaneous area.

49. Use according to claim 45, wherein said non-toxic dose is 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm.

50. A method of treating at least one penetratable cutaneous area of a human or animal before, during, and/or after penetration of said area, to connect the vascular system of said human or animal with a sampling, infusion, or withdrawal container wound, comprising applying a device to said penetratable cutaneous area, wherein the device elutes nitric oxide (NO) thereto, and thereby exposes said at least one penetratable cutaneous area of said human or animal to said nitric oxide.

51. The method according to claim 50, wherein said penetratable cutaneous area is a head, face, neck, shoulder, back, arm, hand, stomach, genital, thigh, leg, or foot, of a body, and wherein said method comprises applying a patch/pad, tape/coating, dressing, sheath/plaster, gel, hydrogel, foam, spray, or cream to said head, face, neck, shoulder, back, arm, hand, stomach, genital, thigh, leg, or foot, of a body, for said exposure.

52. The method according to claim 50, wherein said exposure to nitric oxide (NO) is obtained by a NO eluting polymer.

53. The method according to claim 52, wherein release of NO from the NO eluting polymer is regulated or controlled by a carrier material.

54. The method according any of claims 50, wherein said nitric oxide (NO) obtains a vasodilating, anti-contraction and anti-spasm effect at said penetratable cutaneous area.

55. The method according to any of claims 50, comprising substantially directing said elution of said nitric oxide (NO) from said device towards said penetratable cutaneous area for said exposure for obtaining a vasodilating, anti-contraction and anti-spasm effect at said penetratable cutaneous area.
